(19) United States
(12) Patent Application Publication
(10) Pub. No.: US 2011/0092564 A1
(43) Pub. Date: Apr. 21, 2011

(54) CYCLIC AMINO ACIDS FOR THE TREATMENT OF PAIN

(76) Inventors: Ernest Puil, Vancouver (CA); Bernard A. Macleod, Vancouver (CA); Richard Wall, Vancouver (CA)

(21) Appl. No.: 12/919,657

(22) PCT Filed: Feb. 26, 2009
(86) PCT No.: PCT/CA2009/000237

§ 371 Date: Dec. 6, 2010

Related U.S. Application Data
(60) Provisional application No. 61/066,983, filed on Feb. 26, 2008.

(51) Int. Cl. A61K 31/198; C07C 229/48; A61P 29/00; C07D 313/04; A61K 31/335; A61K 31/351; A61K 31/34; A61K 31/337; C07D 309/14; C07D 307/24; C07D 305/08; C07D 337/04; C07D 335/02; C07D 333/38; C07D 331/04; A61K 31/382; A61K 31/381; A61K 31/38

(52) U.S. Cl. 514/431; 562/505; 514/561; 549/346; 514/450; 514/459; 514/472; 514/449; 549/424; 549/480; 549/510; 549/9; 549/28; 549/68; 549/88; 514/432; 514/447; 514/430

(57) ABSTRACT

Compositions comprising cyclic amino acids or pharmaceutically acceptable salts thereof and a pharmaceutically acceptable carrier. Said composition are for use in the treatment of pain. Pain includes both acute and chronic forms of pain. The preferred cyclic amino acid is 1-aminocyclobutane-1-carboxylic acid (ACBC).

### TECHNICAL FIELD OF THE INVENTION

This invention relates to therapeutic compounds and compositions for treating pain.

### BACKGROUND AND SUMMARY OF THE INVENTION

There is a need to provide new analgesic drugs, which have improved properties such as lower toxicity and a decreased incidence of side-effects. Pain may generally be classified as acute or chronic. Acute pain begins suddenly and is short-lived (usually in twelve weeks or less). In contrast, chronic pain is long-term affair, typically persisting for more than three months and leading to significant psychological and emotional problems.

Neuropathic pain currently is defined as pain initiated or caused by a primary lesion, or resulting in a dysfunction in the nervous systems. Trauma and disease commonly cause nerve damage and hence the term “neuropathic pain” encompasses many disorders with diverse aetiologies. These conditions include, but are not limited to, peripheral, diabetic, HIV, and cancer neuropathies, posttherapeutic and trigeminal neuralgias, back pain, phantom limb pain, carpal tunnel syndrome, and pain associated with chronic alcoholism, epilepsy, hypothyroidism, multiple sclerosis, Parkinson's disease, spinal cord injury, central stroke, uremia, and vitamin deficiency.

In the CNS, modulation of pain can occur at the level of the spinal cord, brainstem, thalamus and neocortex. The activity generated by nociceptor input in the periphery produces a primary afferent input to neurons in the dorsal horn of the spinal cord. Inhibitory modulation is possible at this site by virtue of receptors for several neurotransmitters like GABA and glycine. Endowed with opioid receptors, this synaptic site has a well-known susceptibility to modulation by opioid analgesic drugs. The second order neurons process and send the information directly to thalamic and brainstem neurons. Brainstem neurons also are heavily endowed with receptors for opioids which modulate pain, as well as their toxic effects. Brainstem neurons also engage in physiological modulation of spinal nociception, by virtue of descending influences that result in synaptic inhibition of spinal neurons. Brainstem neurons relay nociceptive information to nuclei of the ventrobasal thalamus, and more diffusely, to the neocortex where modulation is possible. On complex processing by the thalamic neurons, the nociceptive information is sent to the neocortex which generates perception of pain.

Pharmacological intervention is feasible at each of the four levels of the neuraxis, but particularly where nociceptive impulses converge at the major gateways—the spinal cord and thalamus. Arising from the dorsal spinal cord, the medial lemniscal pathway conveys somatosensory information from the periphery and provides the thalamus with nociceptive information. Spinothalamic and trigeminothalamic pathways exclusively convey nociceptive information. The pathways ascend from the spinal cord without synapsing, and diffusely terminate in the ventrobasal complex of the thalamus. After processing, the neurons relay the new patterns of impulses to the neocortex where processing of the received information acquires a context of pain. Thalamocortical neurons of the midline and ventroposterolateral nuclei engage in substantial processing of their nociceptive inputs from the neocortex. The ongoing impulse-dialogue between neocortex and thalamus results in the perception of pain.

A large number of potential targets exist for analgesic drugs including sodium, potassium and calcium ion channels as well as receptor systems. The presynaptic and postsynaptic receptors include receptors for neutral amino acids such as glutamate GABA (gamma amino butyric acid) and glycine, opioids, cannabinoids, prostaglandins, serotonin (5-HT), norepinephrine, purines and nicotine. Several pharmacologically distinct classes are routinely used to treat acute and chronic pain. These include opioids, nonsteroidal antiinflammatory drugs (NSAIDs), anticonvulsants and antimigraine drugs (5-HT receptor agonists and ergot derivatives). Morphine is a standard reference analgesic that acts on the central nervous system. NSAIDs and anti-migraine drugs act primarily on peripheral tissues.

Acute pain may be treated by drugs belonging to the opioid and NSAID classes. Major side effects limit the clinical efficacy of these drugs. In addition the short duration of most drugs necessitates repeated dosing.

Both opioids and NSAIDs have dangerous side effects. Opioids result in respiratory depression, a life threatening side effect. NSAIDs produce gastrointestinal bleeding and renal damage.

While 5-HT agonists and ergot derivatives (antimigraine medications) are efficacious the analgesic responses produced are highly variable. Without being bound by theory, it is believed that their complex pharmacology can account for the variable responses and appreciable side effects (such as nausea and vomiting).

Chronic pain is poorly responsive to opioids and NSAIDs. Opioids may diminish chronic pain of neuropathic and psychogenic origins, unfortunately at doses that have unacceptable side effects.

Antidepressant agents, such as the tricyclics (e.g., nortryptyline) reduce moderate neuropathic and psychogenic pain. Anticonvulsants (e.g., gabapentin), sodium channel blockers (e.g., lidocaine) anxiolytics, or glucocorticoids, can be used alone or in combination with opioids or NSAIDs. Long term use of these combinations is limited by side effects. Opioids are associated with tolerance and dependence, whereas NSAIDs produce gastrointestinal bleeding, and tricyclic antidepressants are associated with hypotension, sedation and weight gain.

In one embodiment, symptomatic treatment of pain or pain syndromes is described using compounds of formula I

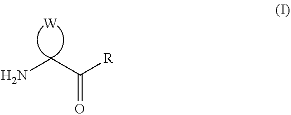

or a pharmaceutically acceptable salt thereof;

wherein

W is C₃-C₇ alkylene, C₂-C₆ heteroalkylene, C₃-C₇ unsaturated alkylene or C₂-C₆ unsaturated heteroalkylene, each of which is optionally substituted; and

R is OH or NX¹X²; where X¹ is hydrogen or C₁-C₆ alkyl; and X² is hydrogen or C₁-C₆ alkyl and wherein the compound relieves pain. In one embodiment, the compound consists essentially of the compound of formula I. In other embodiments, the compound of formula II) may not be linked to another pain-relieving compound, 2) may relieve pain through a mechanism distinct from the mechanism of morphine or NSAIDs, or 3) at high analgesic doses may produce no discernible sedation or respiratory depression.

In one embodiment, the inventors have shown that the compounds of formula I, as further described below, are effective in treating pain when administered in established animal models of pain. For example, the compounds of formula I may have similar effectiveness to morphine in suppressing the acute and chronic phases of pain generated by injecting formalin into the mouse hind-paw. Compounds of formula I can produce analgesia in chronic pain models.

In one embodiment, a composition comprising a compound of the formula

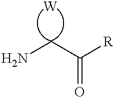

or a pharmaceutically acceptable salt thereof, for the treatment of pain is described;

wherein

W is C₃-C₇ alkylene, C₂-C₆ heteroalkylene, C₃-C₇ unsaturated alkylene or C₂-C₆ unsaturated heteroalkylene, each of which is optionally substituted; and

R is OH or NX¹X²; where X¹ is hydrogen or C₁-C₆ alkyl; and X² is hydrogen or C₁-C₆ alkyl.

In another embodiment, the preceding embodiment is described wherein W and the attached carbon form an optionally substituted carbocycle. In another embodiment, the preceding embodiment is described wherein the carbocycle is cyclobutyl. In another embodiment, any one of the preceding embodiments is described wherein W and the attached carbon form an optionally substituted heterocycle.

In another embodiment, any one of the preceding embodiments is described wherein W and the attached carbon form a group of the formula

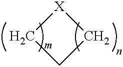

wherein X is selected from the group consisting of optionally substituted methylene, oxygen, sulfur, sulfinyl, or sulfonyl, NR¹, and R²C═CR³, where R¹ is hydrogen, alkyl, heteroalkyl or acyl; and R² and R³ are in each instance independently selected from the group consisting of hydrogen, alkyl, and heteroalkyl, or R² and R³ are taken together with the attached carbons to form an optionally substituted cycle; m is 1 to 5; and n is 1 to 5, where m+n is 2 to 6. In another embodiment, the preceding embodiment wherein X is oxygen or sulfur is described. In another embodiment, any of the preceding embodiments wherein X is oxygen is described. In another embodiment, any of the preceding embodiments wherein X is sulfur is described. In another embodiment, any of the preceding embodiments wherein X is NR¹ and R¹ is hydrogen or alkyl is described. In another embodiment, any of the preceding embodiments wherein R is OH is described. In another embodiment, any of the preceding embodiments is described wherein the composition of any one of claims 1 to 9 further comprises one or more pharmaceutically acceptable excipients, diluents or carriers therefor, or a combination thereof.

In one embodiment, the compound consists essentially of the compound of formula I. In any of the preceding embodiments, the compound of formula II) may not be linked to another pain-relieving compound, 2) may relieve pain through a mechanism distinct from the mechanism of morphine or NSAIDs, or 3) at high analgesic doses may produce no discernible sedation or respiratory depression.

In yet another embodiment, a method for treating a patient in need of relief from pain is provided. The method comprises the step of administering to the patient a therapeutically effective amount of the compound or composition of any of the preceding embodiments. In another embodiment, the preceding embodiment wherein the pain is acute pain or chronic pain is described. In another embodiment, any one of the preceding embodiments wherein the pain is neuropathic pain is described. In another embodiment, any of the preceding embodiments is described wherein the pain results from one or more causes selected from the group consisting of a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms.

In another embodiment, any one of the embodiments described herein wherein the composition is adapted for oral, inhalation, topical, rectal, vaginal, or parenteral administration is described. In another embodiment, any of the embodiments described herein, wherein the composition is adapted for subcutaneous, intramuscular, intravenous, or intrathecal administration is described.

In another embodiment, any of the preceding embodiments wherein the patient is a human or a non-human mammal is described. In another embodiment, any one of the preceding embodiments wherein the patient is a human is described. In another embodiment, any of the preceding embodiments wherein the treatment is of a domestic animal or an animal in captivity is described.

In another embodiment, the use of any one or a combination of more than one of the compositions of any of the preceding embodiments for the manufacture of a medicament for the treatment of pain is described. In another embodiment, the preceding embodiment wherein the pain is acute pain or chronic pain is described. In another embodiment, any of the preceding medicament embodiments wherein the pain is neuropathic pain is described.

In another embodiment, any of the preceding medicament embodiments wherein the pain results from one or more causes selected from the group consisting of a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms is described.

In another embodiment, any of the preceding embodiments wherein the medicament is adapted for oral inhalation, topical, rectal, vaginal, or parenteral administration is described. In another embodiment, any of the preceding embodiments wherein the medicament is adapted for parenteral administration, where the parenteral administration is selected from the group consisting of subcutaneous, intramuscular, intravenous and intrathecal administration is described.

In another embodiment, the composition of any of the embodiments described herein wherein the pain is acute pain or chronic pain is described. In another embodiment, the composition of any of the embodiments described herein wherein the pain is neuropathic pain is described. In another embodiment, the composition of any of the embodiments described herein wherein the pain results from one or more causes selected from the group consisting of a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms is described.

In another embodiment, a kit comprising the compound or composition of any one of the preceding claims in an amount effective to relieve pain and instructional materials describing how to use the kit wherein the instructional materials indicate that the compound or composition should be used for treating pain is described. In another embodiment, the preceding embodiment wherein the composition is in a form adapted for oral inhalation, topical, rectal, vaginal, or parenteral administration is described. In another embodiment, the kit of any of the preceding embodiments further comprising an additional pain treatment compound selected from the group consisting of opioids, non-steroidal anti-inflammatory drugs, anticonvulsants and anti-migraine drugs is described.

In one embodiment, the compound consists essentially of the compound of formula I. In any of the preceding embodiments, the compound of formula II) may not be linked to another pain-relieving compound, 2) may relieve pain through a mechanism distinct from the mechanism of morphine or NSAIDs, or 3) at high analgesic doses may produce no discernible sedation or respiratory depression.

### BRIEF DESCRIPTION OF THE DRAWINGS

FIG. 1. Depressant effects of 1-amino-cyclobutane-1-carboxylic acid (ACBC) on tonic firing in ventrobasal thalamus. ACBC (1 μM) depressed tonic firing evoked by current pulse injection. This inhibition could be overcome with a larger current step, as shown in the lower recording during ACBC application (1 μM). ACBC did not greatly affect the configuration of action potentials. Recordings were made at a holding potential of −60 mV.

FIG. 2. ACBC Reduced Input Resistance (Ri) And Membrane Potential (Vm) In Ventrobasal Thalamus. Ri () and Vm (◯), normalized to the control (5 minutes prior to drug application), both decreased after application of 1 μM ACBC. Measurements are from a single neuron. The arrow indicates the application of ACBC, which occurred at 0 minutes.

FIG. 3. Current-Voltage Relationships For ACBC And Glycine In Ventrobasal Thalamus. Panel A: ACBC (, 1 μM) increased the slope of the I-V curve which intersected the control (◯, aCSF) curve at −92 mV, between chloride equilibrium potential of −53 and potassium's equilibrium potential of −104 mV. Panel B: Glycine (◯, 150 μM) increased the slope of the I-V curve which intersected the control curve at −53 mV, which is chloride's equilibrium potential. The membrane potential at the intersection between the control curve (, aCSF) and the drug curve is taken as the equilibrium potential for the drug.

FIG. 4: ACBC Induces An Outward Current. When voltage-clamped at −60 mV, the holding current increased in magnitude from 33 pA during the control (5 minutes before onset of a 3 min, application of ACBC), to 153 pA 20 minutes after ACBC (, 1 μM) application.

FIG. 5. Model of Acute Pain. Intrathecal ACBC greatly reduces the phase I licking in formalin foot mice. ACBC=Morphine<control P,<0.05 N=5

FIG. 6. Model of Chronic Pain: Intrathecal ACBC greatly reduces the phase II licking in formalin foot mice (comparable to morphine). ACBC<control, P,<0.05 N=5

FIG. 7. Treatment with ACBC Yielding 60% Block Of Chronic Pain. Using the up and down method the ED50 was found to be 94±10 mg/kg subcutaneously.

### DESCRIPTION OF ILLUSTRATIVE EMBODIMENTS

In one embodiment, symptomatic treatment of pain or pain syndromes is described using compounds of formula I

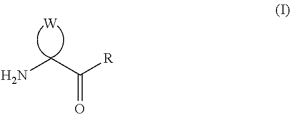

or a pharmaceutically acceptable salt thereof;

wherein

W is C₃-C₇ alkylene, C₂-C₆ heteroalkylene, C₃-C₇ unsaturated alkylene or C₂-C₆ unsaturated heteroalkylene, each of which is optionally substituted; and

R is OH or NX¹X²; where X¹ is hydrogen or C₁-C₆ alkyl; and X² is hydrogen or C₁-C₆ alkyl and wherein the compound relieves pain. In one embodiment, the compound consists essentially of the compound of formula I. In other embodiments, the compound of formula II) may not be linked to another pain-relieving compound, 2) may relieve pain through a mechanism distinct from the mechanism of morphine or NSAIDs, or 3) at high analgesic doses may produce no discernible sedation or respiratory depression.

In some embodiments, the treatment of acute (e.g. postoperative pain), recurring episodic pain, and episodic or persistent chronic pain that are difficult to treat with existing drugs, or are problematic due to toxicity or side effects is described. Acute pain is pain that comes on quickly but lasts a relatively short time. Neuropathic pain amenable to treatments of the invention encompasses an ensemble of chronic pain syndromes that originate from one or more lesions and/or dysfunctions in the central or peripheral nervous systems.

In alternative aspects, the present invention pertains to the treatment of episodic disorders such as migraine headache, and other pain syndromes which are severe and deeply felt, stabbing or lancing, insidious and dull, pressure-like with a sensation of distension, or intense and deeply felt as if produced by a sharp boring instrument.

The terms “analgesic medication”, “analgesic effect” or “analgesia”, mean drugs, or the effects of drugs, that reduce or suppress pain without unduly affecting other sensory modalities and conscious motor behavior. Accordingly, in some aspects of the invention, compounds of formula I are used to reduce, prevent, or block acute and chronic pain in patients without undue side effects.

In various aspects of the invention, the administration of compounds of formula I may be carried out so as to produce a dose-dependent reduction in the intensity of pain symptoms. The pain symptoms may for example be objectified by the measurement of different behaviour and/or conscious physiological parameters such as vocalization, inordinate responses to normal sensory stimulation, etc. The analgesic effect may be maintained in as much as the circulating amounts of the analgesic compound are adjusted by dosing so as to be sufficient for exerting its analgesic activity.

In some embodiments, in neurons of nociceptive nuclei compounds of formula I have a reversal potential between equilibrium potentials for chloride and potassium ions. Thus, compounds of formula I may increase membrane conductance for potassium leading to decreased excitability to electrical and chemical stimuli. This receptor activation by compounds of formula I may decrease excitability of neurons in the nociceptive system of the CNS, thereby modulating pain transmission and producing the analgesic effects.

In various embodiments, many types of chronic pain of neuropathic and psychogenic origin are potentially sensitive to the action of the compounds and compositions described herein. In one embodiment, pain is considered chronic when a patient has begun to suffer for a period of 3 months. In one aspect, the invention involves administration (including co-administration) of therapeutic compounds or compositions, such as 1-aminocyclobutane-1-carboxylic acid of formula I, and the corresponding salts of addition with acids or bases, so as to achieve analgesia in a patient. In various embodiments, such agents may be used therapeutically in compositions, formulations or medicaments. Accordingly, the invention provides therapeutic compositions comprising active agents, including analgesic agents that achieve an analgesic effect in a patient, together with pharmacologically acceptable carriers, excipients or diluents.

In alternative embodiments, the compounds of formula I may be formulated for oral, rectal, vaginal, parenteral (including subcutaneous, intramuscular, intravenous and intrathecal), or topical administration.

In various embodiments, patients may be selected for treatment, or treated, so that the amount of a compound of formula I used is effective to provide analgesia without the undesirable side effects of morphine at conventional doses, the effective amount of a compound of formula I is effective to have an analgesic effect in the subject but is not effective to treat any organic pathology in the subject, or the compounds of formula I are present in a therapeutically effective amount but not in an amount in which the isovaline is an effective adjuvant, excipient or stabilizer for a co-formulated active ingredient.

In alternative aspects, the pain to be treated may result from one or more causes, such as a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms.

In various embodiments, pharmaceutically acceptable salts of compounds of formula I include the acid addition and base salts thereof.

Suitable acid addition salts are formed from acids which form non-toxic salts. Non-limiting examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Non-limiting examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

In one embodiment, a compound of formula I may exist in both unsolvated and solvated forms. The term “solvate” is used herein to describe a molecular complex comprising the compound of formula I and a stoichiometric amount of one or more pharmaceutically acceptable solvent molecules, for example, ethanol. The term “hydrate” is employed when said solvent is water.

In various embodiments, included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J. Pharm. Sci., 64 (8), 1269-1288, by Haleblian (August 1975).

In various embodiments, references to a compound of formula I can include salts, solvates and complexes thereof and solvates and complexes of salts thereof.

In another embodiment, a compound of formula I, as herein defined, may exist in one or more crystalline (polymorphic) or isomeric forms (including optical, geometric and tautomeric isomers), in an isotopically labelled form or as a prodrug. Such crystalline/isomeric forms and prodrugs are within the scope of the present invention and are further described below.

In other illustrative aspects, metabolites of compounds of formula I, that is, compounds formed in vivo upon administration of the drug are provided. Some non-limiting examples of metabolites, in accordance with the invention include, the hydroxylated compound formed from a compound of formula I wherein W and the attached carbon form a cyclobutyl ring, where a methylene group of the cyclobutyl ring is modified to form a hydroxy-substituted methylene group (—CH₂→—CHOH).

Examples of isotopes suitable for inclusion in the compounds of the invention include isotopes of hydrogen, such as ²H and ³H, carbon, such as ¹¹C, ¹³C and ¹⁴C, nitrogen, such as ¹³N and ¹⁵N, oxygen, such as ¹⁵O, ¹⁷O and ¹⁸O. It is appreciated that isotopically-labelled compounds of formula I, for example, those incorporating a radioactive isotope, are useful in drug and/or substrate tissue distribution studies. The radioactive isotopes tritium, i.e. ³H, and carbon-14, i.e. ¹⁴C, are useful for this purpose in view of their ease of incorporation and ready means of detection.

Without being bound by theory, it is believed that substitution with heavier isotopes such as deuterium, i.e. ²H, may afford certain therapeutic advantages resulting from greater metabolic stability, for example, increased in vivo half-life or reduced dosage requirements.

In another embodiment, substitution with positron emitting isotopes, such as ¹¹C, ¹⁵O and ¹³N, can result in compounds useful in Positron Emission Topography (PET) studies for examining substrate receptor occupancy.

Isotopically-labeled compounds of formula I can generally be prepared by conventional techniques known to those skilled in the art or by processes analogous to those described in the accompanying Examples using an appropriate isotopically-labeled reagent in place of the non-labeled reagent.

In one embodiment, pharmaceutically acceptable solvates in accordance with the invention include those wherein the solvent of crystallization may be isotopically substituted, e.g. D₂O, d₆-acetone, d₆-DMSO.

The compounds of formula I can be prepared by any conventional route or procedure known to those skilled in the art of organic synthesis from precursors that are commercially available.

In one illustrative aspect, compounds of formula I may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

In various embodiments, the compounds of formula I may be administered alone or in combination with one or more other compounds of formula I or in combination with one or more other drugs (or as any combination thereof) other than another pain relief medication that is linked to the compound of formula I. In one embodiment, the compounds of formula I will be administered as a formulation in association with one or more pharmaceutically acceptable excipients. The term “excipient” is used herein to describe any ingredient other than a compound of formula I. The choice of excipient will to a large extent depend on factors such as the particular mode of administration, the effect of the excipient on solubility and stability, and the nature of the dosage form.

Pharmaceutical compositions suitable for the delivery of compounds of formula I and methods for their preparation will be readily apparent to those skilled in the art. Such compositions and methods for their preparation may be found, for example, in Remington's Pharmaceutical Sciences, 19th Edition (Mack Publishing Company, 1995).

In various embodiments, the pharmaceutical compositions containing the active ingredient may be in a form suitable for oral use, for example, as tablets, troches, liquids, lozenges (including liquid-filled lozenges), chews, multi- and nano-particulates, gels, aqueous or oily suspensions, dispersible powders or granules, emulsions, hard or soft capsules, films, ovules, sprays, or syrups or elixirs. Oral administration may involve swallowing, so that the compound enters the gastrointestinal tract, or buccal or sublingual administration may be employed by which the compound enters the blood stream directly from the mouth. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide a pharmaceutically palatable preparation.

In other embodiments, formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil.

In various embodiments, liquid formulations may include suspensions, solutions, syrups and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

In one embodiment, an aqueous suspension may contain the active materials in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ethyl, n-propyl, or p-hydroxybenzoate; one or more coloring agents; one or more flavoring agents; and one or more sweetening agents such as sucrose or saccharin.

In one embodiment, an oily suspension may be formulated by suspending the active ingredient in a vegetable oil, for example, arachis oil, olive oil, sesame oil or coconut oil, or in a mineral oil such as liquid paraffin. The oily suspension may contain a thickening agent, for example, beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

In one illustrative embodiment, dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

In another embodiment, the pharmaceutical compositions of the invention may also be in the form of oil-in-water emulsions. The oily phase may be a vegetable oil, for example, olive oil or arachis oils, or a mineral oil, for example, liquid paraffin or mixtures of these. Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate. The emulsions may also contain sweetening and flavoring agents.

In yet another embodiment, a compound of formula I may also be used in fast-dissolving, fast-disintegrating dosage forms know to a person of skill in the art of drug dosage forms.

In various illustrative embodiments, for tablet dosage forms, depending on dose, a compound of formula I may make up from 1 weight % to 80 weight % of the dosage form, more typically from 5 weight % to 60 weight % of the dosage form. In addition to the compound of formula I, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Typically, the disintegrant will comprise from 1 weight % to 25 weight %, preferably from 5 weight % to 20 weight % of the dosage form.

In other embodiments, binders are used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

In another embodiment, tablets can contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

In other embodiments, possible ingredients include anti-oxidants, colorants, flavoring agents, preservatives and taste-masking agents.

Exemplary tablets contain up to about 80% of a compound of formula I, from about 10 weight % to about 90 weight % binder, from about 0 weight % to about 85 weight % diluent, from about 2 weight % to about 10 weight % disintegrant, and from about 0.25 weight % to about 10 weight % lubricant.

In one embodiment, tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated or encapsulated. The methods for the formulation of tablets are known to those skilled in the art of drug delivery forms.

In one illustrative aspect, consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

In one aspect, a compound of formula I for use in a film may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, a compound of formula I may be used in the form of multiparticulate beads.

In another aspect, the film-forming polymer may be selected from natural polysaccharides, proteins, or synthetic hydrocolloids and is typically present in the range 0.01 to 99 weight %, more typically in the range 30 to 80 weight %.

Other possible ingredients in such a film include anti-oxidants, colorants, flavorings, flavor enhancers, preservatives, salivary stimulating agents, cooling agents, co-solvents (including oils), emollients, bulking agents, anti-foaming agents, surfactants and taste-masking agents.

In one illustrative aspect, films in accordance with the invention are typically prepared by evaporative drying of thin aqueous films coated onto a peelable backing support or paper. This may be done in a drying oven or tunnel, typically a combined coater dryer, or by freeze-drying or vacuuming.

In another embodiment, solid formulations for oral administration may be formulated to be immediate and/or modified release. Modified release formulations include delayed, sustained, pulsed, controlled, targeted and programmed release formulations. Suitable modified release formulations for the purposes of the invention are described in U.S. Pat. No. 6,106,864. Details of other suitable release technologies such as high energy dispersions and osmotic and coated particles are known to persons of ordinary skill in the art (Verma, et al., 2001). The use of chewing gum to achieve controlled release is described in WO-A-00/35298.

In one aspect, a compound of formula I may also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular and subcutaneous delivery. Suitable means for parenteral administration include needle (including microneedle) injectors, needle-free injectors and infusion techniques.

In one illustrative aspect, parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. In one embodiment, the solubility of a compound of formula I used in the preparation of a parenteral formulation may be increased by the use of appropriate formulation techniques, such as the incorporation of solubility-enhancing agents.

In various embodiments, formulations for parenteral administration may be formulated to be for immediate and/or modified release. Modified release formulations include delayed, sustained, pulsed, controlled, targeted and programmed release formulations. Thus, a compound of formula I may be formulated as a solid, semi-solid, or thixotropic liquid for administration as an implanted depot providing modified release of the active compound. Examples of such formulations include drug-coated stents and copolymeric dl-lactic, glycolic)acid (PGLA) microspheres. In another embodiment, compounds of formula I or compositions comprising compounds of formula I may be continuously administered, where appropriate.

In yet another embodiment, a compound of formula I may be administered topically to the skin or mucosa, i.e. dermally or transdermally. Typical formulations for this purpose, according to methods recognized in the art, include gels, hydrogels, lotions, solutions, creams, ointments, suspensions, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. In other embodiments, penetration enhancers known to those skilled in the art of drug delivery methods may be incorporated

In other embodiments, means of topical administration include delivery by electroporation, iontophoresis, phonophoresis, sonophoresis and microneedle or needle-free (e.g. Powderject™, Bioject™, and the like.) injection.

In these aspects, formulations for topical administration may be formulated to be for immediate and/or modified release. Modified release formulations include delayed, sustained, pulsed, controlled, targeted and programmed release formulations. In yet another embodiment, a compound of formula I can be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser may contain a solution or suspension of a compound of formula I comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

In one embodiment, prior to use in a dry powder or suspension formulation, a drug product can be micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

In another illustrative aspect, capsules (made, for example, from gelatin or hydroxypropyl-methylcellulose), blisters and cartridges for use in an inhaler or insufflator may be formulated to contain a powder mix of a compound of formula I, a suitable powder base such as lactose or starch and a performance modifier such as l-leucine, mannitol, or magnesium stearate. The lactose may be anhydrous or in the form of the monohydrate. Other suitable excipients include dextran, glucose, maltose, sorbitol, xylitol, fructose, sucrose and trehalose.

In one illustrative aspect, a suitable solution formulation for use in an atomiser using electrohydrodynamics to produce a fine mist may contain from about 1 μg to about 20 mg of a compound of formula I per actuation and the actuation volume may vary from about 1 μl to about 100 μl. A typical formulation may comprise a compound of formula I, propylene glycol, sterile water, ethanol and sodium chloride. Alternative solvents which may be used instead of propylene glycol include glycerol and polyethylene glycol.

In one illustrative aspect, suitable flavors, such as menthol and levomenthol, or sweeteners, such as saccharin or saccharin sodium, may be added to those formulations intended for inhaled/intranasal administration. Formulations for inhaled/intranasal administration may be formulated to be for immediate and/or modified release using, for example, PGLA. Modified release formulations include delayed, sustained, pulsed, controlled, targeted and programmed release formulations.

In the case of dry powder inhalers and aerosols, the dosage unit is determined by means of a valve which delivers a metered amount. Units in accordance with the invention are typically arranged to administer a metered dose or “puff”. The overall daily dose will be administered in a single dose or, more usually, as divided doses throughout the day.

In another embodiment, a compound of formula I may be administered rectally or vaginally, e.g. in the form of a suppository, pessary, or enema. These compositions can be prepared by mixing the drug with a suitable non-irritating excipient which is solid at ordinary temperatures but liquid at the internal temperature and will therefore melt to release the drug. Non-limiting examples are cocoa butter or polyethylene glycols. Formulations for rectal/vaginal administration may be formulated to be for immediate and/or modified release. Modified release formulations include delayed, sustained, pulsed, controlled, targeted and programmed release formulations.

In another aspect, a compound of formula I may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

In another embodiment, formulations for ocular/aural administration may be formulated to be for immediate and/or modified release. Modified release formulations include delayed, sustained, pulsed, controlled, targeted, or programmed release formulations.

In one embodiment, a compound of formula I may be combined with a soluble macromolecular entity, such as a cyclodextrin or a suitable derivative thereof or a polyethylene glycol-containing polymer, in order to improve its solubility, dissolution rate, taste-masking, bioavailability and/or stability in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO-A-91/11172, WO-A-94/02518 and WO-A-98/55148.

In various illustrative embodiments, the compound of formula I can be administered in a dose of from about 0.1 mg/kg to about 1000 mg/kg, from about 1.0 mg/kg to about 1000 mg/kg, from about 10 mg/kg to about 1000 mg/kg, from about 50 mg/kg to about 1000 mg/kg, from about 100 mg/kg to about 1000 mg/kg, from about 500 mg/kg to about 1000 mg/kg, from about 1 mg/kg to about 500 mg/kg, from about 1 mg/kg to about 100 mg/kg, from about 1 mg/kg to about 50 mg/kg, from about 1 mg/kg to about 10 mg/kg, from about 5 mg/kg to about 500 mg/kg, from about 10 mg/kg to about 100 mg/kg, from about 20 mg/kg to about 200 mg/kg, from about 10 mg/kg to about 500 mg/kg, or from about 50 mg/kg to about 500 mg/kg, with “kg” referring to kilogram of patient body weight. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

In some embodiments, dosage regimens are adjusted to the individual patient's need and the professional judgment of the person administering or supervising the administration of the compositions. Daily dosages can be administered as a single dose or in multiple doses. For example, the daily dose may be divided into 2, 3, 4 or more equal or unequal doses given at different time intervals during the day. Daytime and night time doses may be different.

As used herein, “treatment” refers to curative, palliative, and prophylactic treatment. Treatment of pain includes eliminating pain, lessening pain, reducing pain, and preventing pain.

In one embodiment, a kit is provided. If a combination of active compounds is to be administered, two or more pharmaceutical compositions may conveniently be combined in the form of a kit suitable for co-administration of the compositions. Such a kit comprises two or more separate pharmaceutical compositions, at least one of which contains a compound of formula I, and means for separately retaining said compositions, such as a container, divided bottle, or divided foil packet. An example of such a kit is the familiar blister pack used for the packaging of tablets, capsules and the like.

In this embodiment, the kit is particularly suitable for administering different dosage forms, for example, oral and parenteral formulations, for administering separate compositions at different dosage intervals, or for titrating separate compositions against one another. To assist compliance, the kit typically comprises directions for administration to treat pain and may be provided with a so-called memory aid.

A “therapeutically effective amount” of a composition comprising the compound of formula I generally refers to an amount effective, at dosages and for periods of time necessary to achieve the desired analgesic result. A therapeutically effective amount of a composition containing the compound of formula I may vary according to factors such as the route of administration, disease state, age, sex, and weight of the individual, sensitivity of the patient, any combination of which may affect the ability of the compound to elicit a desired response in the individual. Dosage regimens may be adjusted to provide the optimum therapeutic response.

In one illustrative aspect, a pharmaceutically acceptable diluent, carrier, or excipient includes any and all solvents, dispersion media, coatings, antibacterial and antifungal agents, isotonic and absorption delaying agents, and the like, and combinations thereof, that are physiologically compatible. In some embodiments, the carrier is suitable for parenteral or intrathecal administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. Supplementary active compounds can also be incorporated into compositions of the invention.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In another illustrative aspect, active agents of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art.

In one embodiment, sterile injectable solutions can be prepared by incorporating the active agent in the required amount in an appropriate solvent with one or a combination of ingredients described above, as required, followed by filtered sterilization. Typically, dispersions are prepared by incorporating the active compound into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In another embodiment, compositions comprising one or more compounds of formula I, in containers having labels that provide instructions for use of, or to indicate the contents as, analgesics, such as compounds to treat, suppress or ameliorate pain, or for treating diseases or disorders listed herein as associated with pain are provided.

In various embodiments, routes of administration include intradermal, transdermal, parenteral, intravenous, intramuscular, intranasal, subcutaneous, regional, percutaneous, intratracheal, intraperitoneal, intraarterial, intravesical, intratumoral, intrathecal, inhalation, perfusion, lavage, direct injection, and oral administration.

Accordingly, compositions of the invention may be adapted to be administered orally, nasally, sublingually, rectally, transcutaneously, parenterally, intrathecally or subarachnoidally. Methods of the present invention may be used prophylactically, for example preoperatively.

In one embodiment, a composition is described comprising a compound of the formula

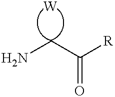

or a pharmaceutically acceptable salt thereof and a pharmaceutically acceptable carrier therefor, for the treatment of pain;

wherein

W is C₃-C₇ alkylene, C₂-C₆ heteroalkylene, C₃-C₇ unsaturated alkylene or C₂-C₆ unsaturated heteroalkylene, each of which is optionally substituted; and

R is OH or NX¹X²; where X¹ is hydrogen or C₁-C₆ alkyl; and X² is hydrogen or C₁-C₆ alkyl.

In another embodiment, the preceding embodiment is described wherein W and the attached carbon form an optionally substituted carbocycle. In another embodiment, the preceding embodiment is described wherein the carbocycle is cyclobutyl. In another embodiment, any one of the preceding embodiments wherein W and the attached carbon form an optionally substituted heterocycle is described.

In one embodiment, the compound consists essentially of the compound of formula I. In other embodiments, the compound of formula II) may not be linked to another pain-relieving compound, 2) may relieve pain through a mechanism distinct from the mechanism of morphine or NSAIDs, or 3) at high analgesic doses may produce no discernible sedation or respiratory depression.

In another embodiment, any one of the preceding embodiments wherein W and the attached carbon form a group of the formula

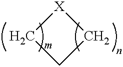

wherein X is selected from the group consisting of optionally substituted methylene, oxygen, sulfur, sulfinyl, or sulfonyl, NR¹, and R²C═CR³, where R¹ is hydrogen, alkyl, heteroalkyl or acyl; and R² and R³ are in each instance independently selected from the group consisting of hydrogen, alkyl, and heteroalkyl, or R² and R³ are taken together with the attached carbons to form an optionally substituted cycle; m is 1 to 5; and n is 1 to 5, where m+n is 2 to 6 is described. In another embodiment, the preceding embodiment wherein X is oxygen or sulfur is described. In another embodiment, any of the preceding embodiments wherein X is oxygen is described. In another embodiment, any of the preceding embodiments wherein X is sulfur is described. In another embodiment, any of the preceding embodiments wherein X is NR¹ and R¹ is hydrogen or alkyl is described. In another embodiment, any of the preceding embodiments wherein R is OH is described. In another embodiment, any of the preceding embodiments wherein the composition of any one of claims 1 to 9 further comprises one or more pharmaceutically acceptable excipients, diluents or carriers therefor, or a combination thereof is described.

In one embodiment, the compound consists essentially of the compound of formula I with the above-described group. In other embodiments, the compound of formula II) may not be linked to another pain-relieving compound, 2) may relieve pain through a mechanism distinct from the mechanism of morphine or NSAIDs, or 3) at high analgesic doses may produce no discernible sedation or respiratory depression.

In yet another embodiment, a method for treating a patient in need of relief from pain is described. The method comprises the step of administering to the patient a therapeutically effective amount of the composition of any of the preceding embodiments. In another embodiment, the preceding embodiment wherein the pain is acute pain or chronic pain is described. In another embodiment, any one of the preceding embodiments wherein the pain is neuropathic pain is described. In another embodiment, any of the preceding embodiments is described wherein the pain results from one or more causes selected from the group consisting of a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms.

In another embodiment, any one of the embodiments wherein the composition is adapted for oral, inhalation, topical, rectal, vaginal, or parenteral administration is described. In another embodiment, any of the embodiments wherein the composition is adapted for subcutaneous, intramuscular, intravenous and intrathecal administration is described.

In another embodiment, any of the preceding embodiments wherein the patient is a human or a non-human mammal is described. In another embodiment, any one of the preceding embodiments wherein the patient is a human is described. In another embodiment for veterinary treatments, any of the preceding embodiments wherein patient is a domestic animal or an animal kept in captivity is described.

In another embodiment, the use any one or combination of more than one of the compositions of any of the preceding embodiments for the manufacture of a medicament for the treatment of pain is described. In another embodiment, the preceding embodiment wherein the pain is acute pain or chronic pain is described. In another embodiment, any of the preceding medicament embodiments wherein the pain is neuropathic pain is described.

In another embodiment, any of the preceding medicament embodiments wherein the pain results from one or more causes selected from the group consisting of a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms is described.

In another embodiment, any of the preceding embodiments wherein the medicament is adapted for oral inhalation, topical, rectal, vaginal, or parenteral administration is described. In another embodiment, any of the preceding embodiments wherein the medicament is adapted for parenteral administration, where the parenteral administration is selected from the group consisting of subcutaneous, intramuscular, intravenous and intrathecal administration is described.

In another embodiment, the composition of any of the embodiments described herein wherein the pain is acute pain or chronic pain is described. In another embodiment, the composition of any of the embodiments wherein the pain is neuropathic pain is described. In another embodiment, the composition of any of the embodiments wherein the pain results from one or more causes selected from the group consisting of a peripheral neuropathy, a central neuropathy, a traumatic abnormality, a cerebral vascular accident, postoperative pain, dental pain, direct trauma, infection, HIV infection, small pox infection, herpes infection, toxic exposure, exposure to arsenic, exposure to lead, cancer, invasive cancer, congenital defect, phantom limb pain, encephalitis, rheumatoid arthritis, fibromyalgias, spinal root lesions, spinal root impingement, back pain, multiple sclerosis, chronic pain, fibrous tissue pain, muscle pain, tendon pain, ligament pain, pain associated with diarrhea, irritable bowel syndrome, abdominal pain, chronic fatigue syndrome, and spasms is described.

In another embodiment, a kit comprising the composition of any one of the preceding claims in an amount effective to relieve pain and instructional materials describing how to use the kit wherein the instructional materials indicate that the composition should be used for treating pain is described. In another embodiment, the preceding embodiment wherein the composition is in a form adapted for oral inhalation, topical, rectal, vaginal, or parenteral administration is described. In another embodiment, the kit of any of the preceding embodiments further comprising an additional pain treatment compound selected from the group consisting of opioids, non-steroidal anti-inflammatory drugs, anticonvulsants and anti-migraine drugs wherein the additional compound not covalently linked to the compound of the invention is described.

Although various embodiments of the invention are disclosed herein, including in the following Examples, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to the present invention. All document cited in this specification are incorporated herein by reference in their entirety as if each individual publication were specifically and individually indicated to be incorporated by reference herein and as though fully set forth herein. The invention includes all embodiments and variations substantially as hereinbefore described and with reference to the examples and drawings.

The method and compositions of the present invention can be used for both human clinical medicine and veterinary applications. Thus, the treated patient can be a human or, in the case of veterinary applications, can be laboratory, agricultural, domestic, or wild animals. The present invention can be applied to animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The compounds described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The compounds described herein may be capable of existing as geometric isomers. Accordingly, it is to be understood that the present invention includes pure geometric isomers or mixtures of geometric isomers.

It is also appreciated that in the foregoing embodiments, certain aspects of the compounds are presented in the alternative, such as selections for any one or more of R, W, X¹, X², R¹, R², R³, m, and n. It is therefore to be understood that various alternate embodiments of the invention include individual members of those lists, as well as the various subsets of those lists. Each of those combinations are to be understood to be described herein by way of the lists.

Heteroalkyl represents a group resulting from the replacement of one or more carbon atoms in a linear or branched alkyl group with an atom independently selected in each instance from the group consisting of oxygen, nitrogen, and sulfur. Some non-limiting examples of heteroalkyl are methoxymethyl, 2-dimethylaminoethyl, 2-(2-dimethylaminoethoxy)ethyl, 2-methylthio-1-propyl, 1-(2-methoxyethoxy)-ethyl, and the like.

Heteroalkylene refers to an alkylene group wherein one or more of the carbons is replaced with a heteroatom. Thus, a C₂-C₆ heteroalkylene group is a straight or branched chain divalent radical that contains from 2 to 6 carbon atoms, and one or more heteroatom independently selected in each instance from the group consisting of optionally substituted nitrogen, oxygen, sulfur, sulfinyl, and sulfonyl.

The term acyl includes formyl, alkylcarbonyl, arylcarbonyl, heteroarylcarbonyl, alkoxycarbonyl, aryloxycarbonyl, heteroaryloxycarbonyl, and carbamoyl, each of which is optionally substituted.

The term “optionally substituted” includes the cases where a group is substituted or unsubstituted. As used herein, “optionally substituted” alkylene, heteroalkylene, unsaturated alkylene and unsaturated heteroalkylene groups may be substituted with zero, or one or more substituents selected from the group consisting of alkyl, aryl, heteroaryl, alkoxy, halo, hydroxy, NO₂, CN, haloalkyl, haloalkoxy, and amino; or with two or more groups wherein two of the groups and the atoms to which they are attached form a cycle.

### EXAMPLES

### Example 1

### Treatment of Thalamic Neurons with ACBC

Current- and voltage-clamp recording techniques were used to determine the effects of ACBC on intrinsic membrane currents of thalamic neurons in in vitro slice preparations, from the brain of Sprague-Dawley rats (13-15 day-old). The procedures for recording and maintenance conditions were similar to those previously described (Wan, Mathers and Puil, Neuroscience 121: 947-958, 2003). Neurons of the ventrobasal nuclei were identified under a differential interference contrast microscope. Whole cell patch-clamp recordings were obtained using an Axoclamp 2A amplifier in both the current- and voltage-clamp modes. Input resistance was measured from <5 mV voltage responses to hyperpolarizing current pulses. In voltage clamp, current-voltage relationships were determined between −50 and −100 mV. ACBC was applied by bath perfusion at 1-2 ml/min.

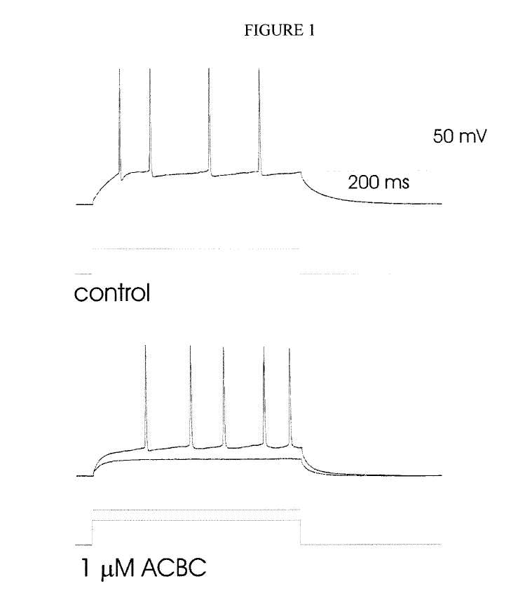

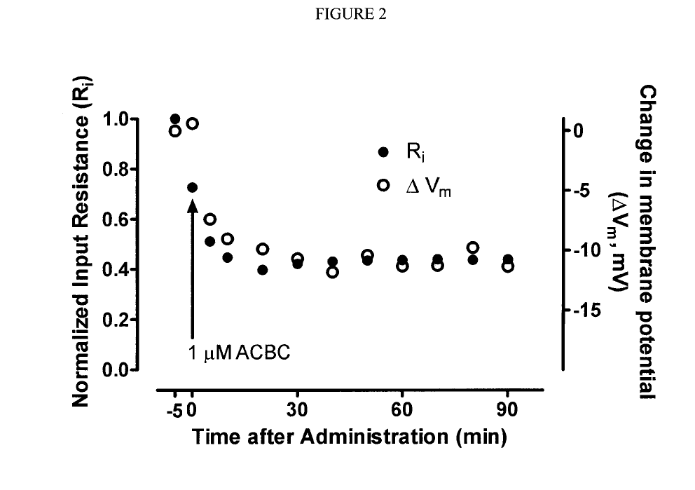

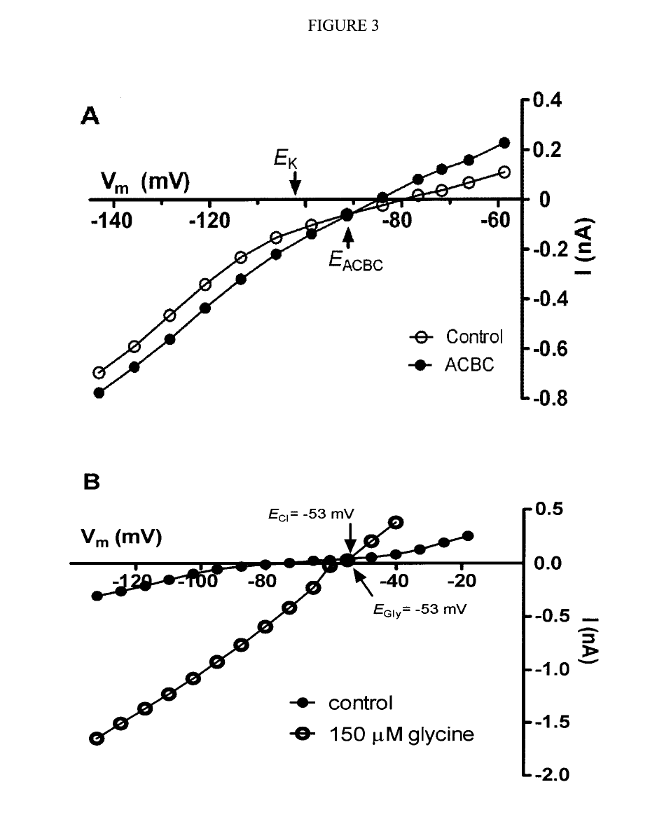

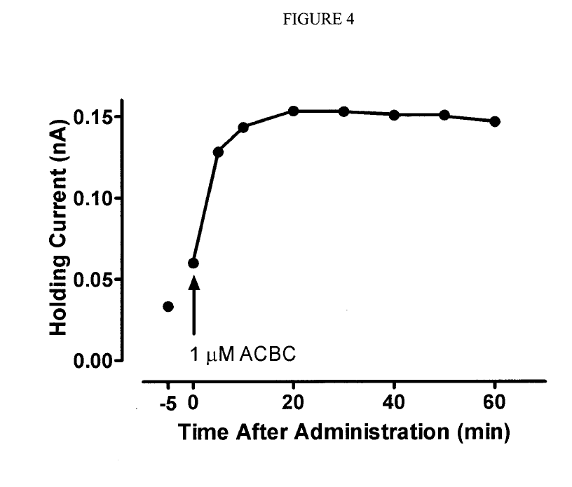

ACBC depressed action potential firing evoked by intracellular injections of current pulses into thalamocortical neurons (FIG. 1). The inhibition of firing is attributable to an increase in membrane conductance since an increase in test current pulse overcame the blockade. In the same neurons, ACBC produced an increase in membrane input resistance (1/conductance; see FIG. 2). Current-voltage relationships showed curves for the control conditions and during application of ACBC that intersected at membrane potentials between the equilibrium potentials of chloride and potassium (see FIG. 3, panel A). The intersection at −92 mV demonstrates a greater contribution of potassium ions than chloride ions to the current. Glycine application alone produced an increase in membrane conductance and a steady outward current attributable to activation of a pure chloride channels (see FIG. 3, panel B). Treatment with ACBC induced an outward current in voltage clamped neurons that persisted for greater than 60 minutes (see FIG. 4).

ACBC decreased membrane excitability and action potential firing of nociceptive neurons of the thalamus. Without being bound by theory it is believed that these effects result from increased membrane conductance to potassium and chloride ions, and the consequent net hyperpolarization. It is unlikely that these actions of ACBC, resulted from antagonism of glutamate receptor activation which would have produced a decreased membrane conductance.

### Example 2

### Effect of Intrathecal Application of ACBC on Peripheral Acute and Chronic Pain

The effects of an example compound (ACBC) of formula I were determined in an acute and chronic peripheral pain model (Kolesnikov, Cristea, Oksman, Torosjan, and Wilson, Brain Research 1029: 217-223, 2004). Groups of mice (n=5) were injected intrathecally into the lumbar segment with ACBC, 5 μL of 250 mM, at 5 minutes prior to subcutaneous formalin injection into the right hindpaw (5% in 20 μL). The mice were monitored for 40 minutes for licking activity and assessed according to the method of Abbott, Franklin, and Westbrook Pain 60; 91-102, 1995. The cumulative licking activity in seconds per 5 minute bin was plotted, comparing the results obtained by treatment with artificial Cerebral Spinal Fluid (aCSF) control or ACBC.

At the same time as the analgesia was accessed, the sedation effects of ACBC were determined in the acute and chronic peripheral pain formalin foot model. Groups of mice (n=5) were injected intrathecally into the lumbar segment with ACBC (5 μL of 250 μM). The mice were monitored for 40 min for sedation. aCSF and morphine controls were also tested. The presence or absence of sedation was determined by an observer blinded to the treatment based on degree of spontaneous activity, posture and response to normal stimuli.

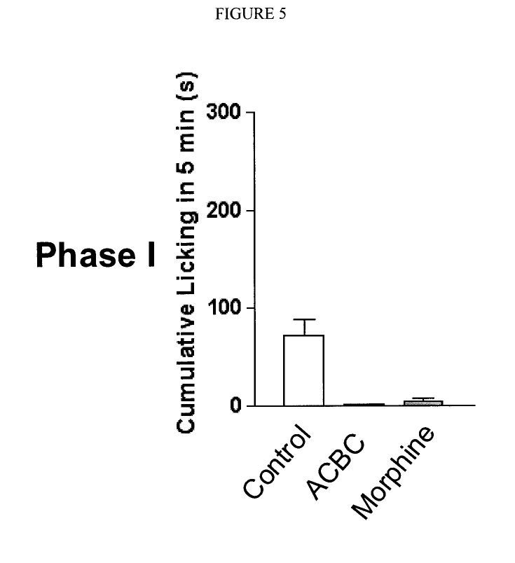

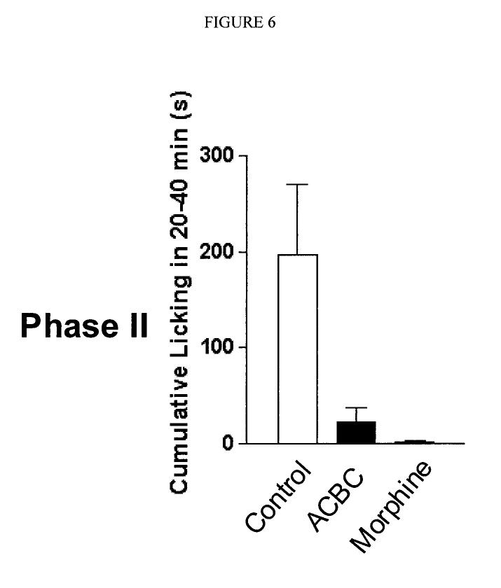

The cumulative paw licking was significantly reduced by ACBC with p-value <0.05. The means (±SEM) of both the early (see FIG. 5, reflecting acute pain) and late (see FIG. 6, reflecting chronic pain) phases were significantly reduced by treatment with ACBC (*p<0.05, compared with the control group). In both the acute and chronic phases the analgesia of ACBC was equal to that produced by morphine.

No sedation occurred during either the acute or the chronic phase of the formalin foot test (see TABLE 1) for aCSF control or for ACBC. However, all mice receiving morphine demonstrated a variety of readily apparent changes in the gross appearance and behavior. The mice generally became nearly immobile within 10 to 20 minutes of injection, although occasional grooming behavior was observed. The animals maintained an upright and somewhat hunched posture with the eyes open, but were considerably less responsive than normal to external stimuli than mice receiving ACBC or aCSF. The results show that ACBC administered to the lumbar cord is active against acute and chronic pain induced peripherally. There was no observed behavioral toxicity of the compound in the animals treated.

TABLE 1

| | Total number of | Number of mice with |
|---|---|---|
| Substance | mice | sedation |
| aCSF | 5 | 0* |
| ACBC | 5 | 0* |
| Morphine | 5 | 5 |

*Significantly different from morphine, P < 0.05

### Example 3

### Subcutaneous Injection of Compound ACBC Reduces Peripheral Acute and Chronic Pain

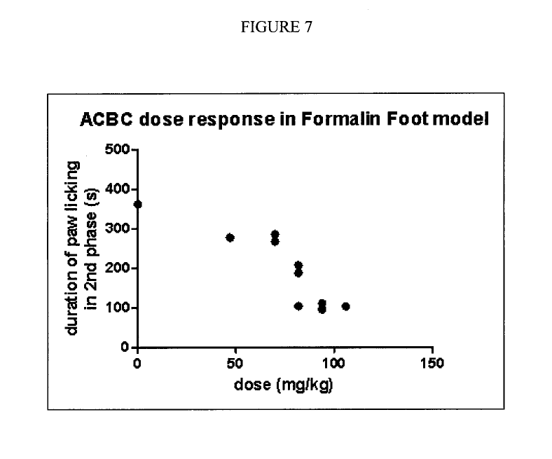

Systemic effects of an example compound (ACBC) of formula I were determined in the acute and chronic peripheral pain model. This model involves pre-treatment with the test compound and injection of formalin (5% in 20 microliters) into the right hindpaw of mice. ACBC was injected subcutaneously 5 minutes prior to formalin injection. The mice were monitored for 40 minutes for licking activity and assessed according to the method of Example 2. The up and down method of Dixon and Mood was used to establish the ED50. Block was defined as a decrease of 60% from controlled cumulated licking, comparing the results obtained with subcutaneous vehicle control to those with ACBC. The ED50 for the reduction of paw licking by 60% was found to be 94 mg/kg with a standard deviation of 10 mg/kg (see FIG. 7).

What is claimed is:

1. A composition comprising a compound of the formula 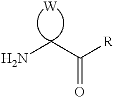
or a pharmaceutically acceptable salt thereof, for the treatment of pain; wherein
W is C₃-C₇ alkylene, C₂-C₆ heteroalkylene, C₃-C₇ unsaturated alkylene or
C₂-C₆ unsaturated heteroalkylene, each of which is optionally substituted; and R is OH or NX¹X¹; where X¹ is hydrogen or C₁-C₆ alkyl; and X′ is hydrogen or C₁-C₆ alkyl, and a pharmaceutically acceptable carrier therefor.

2. The composition of claim 1 wherein W and the attached carbon form an optionally substituted carbocycle.

3. The composition of claim 1 wherein W and the attached carbon form an optionally substituted heterocycle.

4. The composition of claim 2 wherein the carbocycle is cyclobutyl.

5. The composition of claim 1 wherein W and the attached carbon form a group of the formula 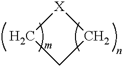
wherein X is selected from the group consisting of optionally substituted methylene, oxygen, sulfur, sulfonyl, or sulfonyl, NR¹, and R²C═CR³, where R¹ is hydrogen, alkyl, heteroalkyl or acyl; and R² and R³ are in each instance independently selected from the group consisting of hydrogen, alkyl, and heteroalkyl, or R² and R³ are taken together with the attached carbons to form an optionally substituted cycle; m is 1 to 5; and n is 1 to 5, where m+n is 2 to 6.

6. The composition of claim 5 wherein X is oxygen.

7. The composition of claim 5 wherein X is sulfur.

8. The composition of claim 5 wherein X is NR¹ and R¹ is hydrogen or alkyl.

9. The composition of claim 1 wherein R is OH.

10. The composition of claim 1 further comprising one or more pharmaceutically acceptable excipients, or diluents, or a combination thereof.

11. A method for treating a patient in need of relief from pain, the method comprising the step of administering to the patient a therapeutically effective amount of the composition of claim 1.

12. The method of claim 11 wherein the pain is acute pain or chronic pain.

13. The method of claim 11 wherein the pain is neuropathic pain.

14.-15. (canceled)

16. The method of claim 11 wherein the composition is adapted for parenteral administration and the type of parenteral administration is selected from the group consisting of subcutaneous, intramuscular, intravenous and intrathecal administration.

17. The method of claim 11 wherein the patient is a human.

18.-23. (canceled)

24. The composition of claim 1 wherein the pain is acute pain or chronic pain.

25. The composition of claims 1 wherein the pain is neuropathic pain.

26.-27. (canceled)

28. The composition of claim 1 wherein the composition is adapted for parenteral administration and the type of parenteral administration is selected from the group consisting of subcutaneous, intramuscular, intravenous and intrathecal administration.

29. A kit comprising the composition of any one of the preceding claims in an amount effective to relieve pain and instructional materials describing how to use the kit wherein the instructional materials indicate that the composition should be used for treating pain.

30. (canceled)

31. The kit of claim 29 further comprising an additional pain treatment not covalently linked to the compound wherein the additional pain treatment is selected from the group consisting of opioids, non-steroidal anti-inflammatory drugs, anticonvulsants, and anti-migraine drugs.
